(19) United States
(12) Patent Application Publication
(10) Pub. No.: US 2020/0339783 A1
(43) Pub. Date: Oct. 29, 2020

(54) FILAMENTS OF MICROFIBRILLATED CELLULOSE

(71) Applicant: Stora Enso OYJ, Helsinki (FI)

(72) Inventors: Gisela Cunha, Nacka (SE); Heidi Saxell, Stockholm (SE)

(73) Assignee: Stora Enso OYJ, Helsinki (FI)

(21) Appl. No.: 16/955,921

(22) PCT Filed: Dec. 20, 2018
(86) PCT No.: PCT/IB2018/060412

§ 371 Date: Jun. 19, 2020

(30) Foreign Application Priority Data
Dec. 21, 2017 (SE) 1751617-0

(51) Int. Cl. C08L 1/02; D21H 11/18; D21H 11/20; D21C 9/00; C08J 5/24; C07C 45/30; D04H 3/015; D01F 2/24

(52) U.S. Cl. CPC C08L 1/02; D21H 11/18; D21H 11/20; D21C 9/005; C08J 5/24; D01D 5/06; D04H 3/015; D01F 2/24; C08J 2301/02; C08L 2205/16; C07C 45/30

(57) ABSTRACT

A method is provided for preparing a fibrous material of crosslinked microfibrillated cellulose. Dialdehyde microfibrillated cellulose is spun into a fibrous material; said fibrous material is pre- or post-treated (by reduction of pH) to provide crosslinking between the dialdehyde microfibrillated cellulose. Fibrous materials such as filaments or mats, and polymer composites comprising such materials are also described.

A method for providing filaments of crosslinked microfibrillated cellulose is provided, as well as spun filaments of crosslinked dialdehyde microfibrillated cellulose. Products comprising said filaments are also described. Such filaments exhibit desirable properties, e.g. strength (in particular wet-strength).

### BACKGROUND

Microfibrillated cellulose (MFC) comprises partly or totally fibrillated cellulose or lignocellulose fibers. The liberated fibrils have a diameter less than 100 nm, whereas the actual fibril diameter or particle size distribution and/or aspect ratio (length/width) depends on the source and the manufacturing methods. The smallest fibril is called elementary fibril and has a diameter of approximately 2-4 nm (see e.g. Chinga-Carrasco, G., Nanoscale research letters 2011, 6:417), while it is common that the aggregated form of the elementary fibrils, also defined as microfibril, is the main product that is obtained when making MFC e.g. by using an extended refining process or pressure-drop disintegration process (see Fengel, D., Tappi J., March 1970, Vol 53, No. 3.). Depending on the source and the manufacturing process, the length of the fibrils can vary from around 1 to more than 10 micrometers. A coarse MFC grade might contain a substantial fraction of fibrillated fibers, i.e. protruding fibrils from the tracheid (cellulose fiber), and with a certain amount of fibrils liberated from the tracheid (cellulose fiber).

There are different acronyms for MFC such as cellulose microfibrils, fibrillated cellulose, nanofibrillated cellulose, fibril aggregates, nanoscale cellulose fibrils, cellulose nanofibers, cellulose nanofibrils, cellulose microfibers, cellulose fibrils, microfibrillar cellulose, microfibril aggregrates and cellulose microfibril aggregates. MFC can also be characterized by various physical or physical-chemical properties such as large surface area or its ability to form a gel-like material at low solids (1-5 wt %) when dispersed in water.

MFC exhibits useful chemical and mechanical properties. Chemical surface modification of MFC has the potential to improve the properties of MFC itself, as well as filaments spun from MFC, e.g. mechanical strength, water absorbance and elasticity/flexibility.

In a recent review article, Lundahl et al. Ind. Eng. Chem. Res., 2017, 56 (1), pp 8-19 provide an overview of methods for spinning MFC into filaments. Among other things, filaments obtained from spinning TEMPO-oxidised MFC are shown to be weaker than filaments spun from non-treated MFC.

An additional problem with common chemically modified MFC, especially if it consists of charged MFC, is that it has increased water absorption when compared to non-modified MFC, due to its chemical charge, and can start losing integrity upon contact with water. Good mechanical strength in wet conditions can therefore be difficult to achieve.

Other documents in this technical field include U.S. Pat. No. 4,256,111 and U.S. Pat. No. 6,027,536.

There therefore remains a need to improve the properties of filaments spun from MFC; in particular, (wet) strength. Suitably, the improvement can be achieved in a straightforward manner, without the use of external modifiers such as crosslinkers.

### SUMMARY

It has been found by the present inventor(s) that fibrous materials (e.g. filaments or webs) with desirable strength, in particular wet strength can be achieved from spinning dialdehyde microfibrillated cellulose (DA-MFC).

A method for preparing a fibrous material (e.g. filaments or mat) of crosslinked microfibrillated cellulose is thus provided, said method comprising the steps of:

i. forming a cellulose composition comprising or consisting of dialdehyde microfibrillated cellulose (DA-MFC) into a fibrous material;

ii. reducing the pH of said fibrous material to pH 7 or below, to provide crosslinking of the dialdehyde microfibrillated cellulose.

A spun fibrous material obtained via the method described herein is also provided, said fibrous material being a spun mat or spun filaments. Additionally, spun fibrous material of crosslinked dialdehyde microfibrillated cellulose, being a spun mat or spun filaments is provided. A web containing such spun filaments is also provided, as is a polymer composite comprising the spun fibrous material. By “spun mat” is mean that—instead of spinning a single filament—one can directly spun an interconnected structure made of filaments.

Further aspects of the invention are provided in the following text and in the dependent claims.

### DETAILED DISCLOSURE

In a first aspect, the invention provides a method for preparing a fibrous material of crosslinked microfibrillated cellulose (MFC). The term “fibrous material” as used herein includes mats and filaments, preferably filaments.

Microfibrillated cellulose (MFC) or so called cellulose microfibrils (CMF) shall in the context of the patent application mean a nano-scale cellulose particle fiber or fibril with at least one dimension less than 100 nm. MFC comprises partly or totally fibrillated cellulose or lignocellulose fibers. The cellulose fiber is preferably fibrillated to such an extent that the final specific surface area of the formed MFC is from about 1 to about 300 m²/g, such as from 1 to 200 m²/g or more preferably 50-200 m²/g when determined for a freeze-dried material with the BET method.

Various methods exist to make MFC, such as single or multiple pass refining, pre-hydrolysis followed by refining or high shear disintegration or liberation of fibrils. One or several pre-treatment step is usually required in order to make MFC manufacturing both energy efficient and sustainable. The cellulose fibers of the pulp to be supplied may thus be pre-treated enzymatically or chemically, for example to reduce the quantity of hemicellulose or lignin. The cellulose fibers may be chemically modified before fibrillation, wherein the cellulose molecules contain functional groups other (or more) than found in the original cellulose. Such groups include, among others, carboxymethyl (CMC), aldehyde and/or carboxyl groups (cellulose obtained by N-oxyl mediated oxidation, for example “TEMPO”), or quaternary ammonium (cationic cellulose). After being modified or oxidized in one of the above-described methods, it is easier to disintegrate the fibers into MFC or NFC.

The nanofibrillar cellulose may contain some hemicelluloses; the amount is dependent on the plant source. Mechanical disintegration of the pre-treated fibers, e.g. hydrolysed, pre-swelled, or oxidized cellulose raw material is carried out with suitable equipment such as a refiner, grinder, homogenizer, colloider, friction grinder, ultrasound sonicator, single—or twin-screw extruder, fluidizer such as microfluidizer, macrofluidizer or fluidizer-type homogenizer. Depending on the MFC manufacturing method, the product might also contain fines, or nanocrystalline cellulose or e.g. other chemicals present in wood fibers or in papermaking process. The product might also contain various amounts of micron size fiber particles that have not been efficiently fibrillated.

MFC can be produced from wood cellulose fibers, both from hardwood or softwood fibers. It can also be made from microbial sources, agricultural fibers such as wheat straw pulp, bamboo, bagasse, or other non-wood fiber sources. It is preferably made from pulp including pulp from virgin fiber, e.g. mechanical, chemical and/or thermomechanical pulps. It can also be made from broke or recycled paper.

The above described definition of MFC includes, but is not limited to, the proposed TAPPI standard W13021 on cellulose nano or microfibril (CMF) defining a cellulose nanofiber material containing multiple elementary fibrils with both crystalline and amorphous regions, having a high aspect ratio with width of 5-30 nm and aspect ratio usually greater than 50.

Dialdehyde microfibrillated cellulose (DA-MFC) is typically obtained by reacting cellulose with an oxidising agent such as periodate. During the periodate oxidation, selective cleavage of the C2-C3 bond of the anhydroglucose (AGU) unit of cellulose takes place, with concurrent oxidation of the C2- and C3-OH moieties to aldehyde moieties. In this manner, crosslinkable functional groups (aldehyde groups) are introduced to the cellulose.

One particular method involves providing a suspension of cellulose pulp fibers in water, and oxidising the cellulose fibers in said water suspension with sodium periodate. Other chemicals that selectively oxidize cellulose in the positions C2 and C3 can also be used, such as periodic acid. After oxidation, oxidised pulp fibers are fibrillated into DA-MFC using any of the known fibrillation processes.

In a first general step of the method, cellulose composition comprising or consisting of dialdehyde microfibrillated cellulose (DA-MFC) is spun into a fibrous material. The fibrous material may be filaments or web.

In the case that the cellulose composition consists of DA-MFC, no components other than DA-MFC are present in the composition. In the case that the cellulose composition comprises DA-MFC, components other than DA-MFC may be present in the composition. However, the cellulose composition suitably comprises more than 25%, preferably more than 50%, such as e.g. more than 75% by weight DA-MFC. In one preferred embodiment, the cellulose composition comprising DA-MFC may additionally comprise unmodified (native) MFC. Suitably, therefore, the cellulose composition consists of DA-MFC and MFC. Alternatively or additionally, the cellulose composition comprising DA-MFC may additionally comprise chemically-modified microfibrillated cellulose, such as e.g. phosphorylated-MFC or TEMPO-MFC (i.e. MFC oxidised with 2,2,6,6-tetramethylpiperidin-1-yl)oxidanyl). For webs, additional components of the cellulose composition may include natural or synthetic filaments or natural or synthetic staple fibres. If a stiff web is obtained, one or more plasticisers may be included.

In a second general step of the method, the pH of the fibrous material from the first step is reduced so as to provide crosslinking of the dialdehyde microfibrillated cellulose. The pH of the fibrous material is reduced prior or after spinning of the material. The pH of the fibrous material is reduced to pH 7 or below. The pH may be reduced to below pH 6.5, suitably below pH 5, preferably below pH 4. Reduction of pH suitably takes place by addition of any suitable acid or buffer.

Exposure of dialdehyde cellulose to neutral or acidic pH gives rise to hemiacetal or acetal groups. Acetal groups are more stable than hemiacetal, and their formation is in reversible equilibrium. In this way, crosslinks are formed directly between the dialdehyde moieties and other components of the cellulose composition.

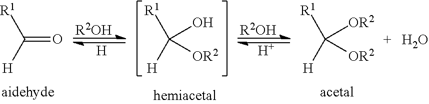

Crosslinking suitably takes place without the use of any additional crosslinking agents; i.e. crosslinks are formed directly between the aldehyde moieties and other components of the cellulose composition.

The removal of water aids in acetal formation, to avoid the conversion of acetal back to aldehyde. An increase in temperature can assist in removing water; therefore the method of the invention may additionally comprise the step of heat-treatment of said fibrous material, suitably concurrently with the step of pH reduction. Heat treatment suitably takes place at a temperature of between 30 and 200° C., preferably between 60-200° C. e.g. between 70 and 120° C. Such temperatures are sufficient to promote crosslinking, but also limit potential degradation of the MFC. Heat treatment suitably takes place for a time of between 10 and 180 minutes, depending on the temperature used and initial solids content of the material to heat treat. Heat treatment may take place in an oven, but other methods of heat treatment may also be used.

The fibrous material is preferably filaments. General methods for spinning filaments from MFC are described e.g. in Lundahl et al. Ind. Eng. Chem. Res., 2017, 56 (1), pp 8-19. Suitable spinning processes may be selected from wet-spinning, electrospinning and dry-spinning. A preferred spinning process for dialdehyde microfibrillated cellulose is wet-spinning, as the coagulation bath from which wet-spinning takes place could itself be an acidic medium. In this situation, coagulation and pH adjustment take place concurrently. The fibrous material may also be a mat.

The general steps of the method (spinning, followed or preceded by pH reduction) may be carried out without any intervening method steps. Alternatively, one or more intervening method steps may be carried out between the spinning step and the pH reduction step.

If hydrated fibrous material is required, a further step of hydrating said fibrous material with water after the pH reduction step may be carried out.

The general method of the invention can be used to provide spun filaments of crosslinked dialdehyde microfibrillated cellulose. The spun filaments can—in turn—be used to prepare a web of spun filaments, by laying said spun filaments to provide a web. The invention therefore provides a web comprising spun filaments, wherein said spun filaments are as described herein.

The web may comprise additional filaments or fibres such as e.g. synthetic filaments, wood fibres or spun filaments of non-modified MFC or other types of modified MFC. The web may be woven or non-woven. The web may be an air-laid, melt-blown or spunlaid non-woven web.

The present invention also provides a spun mat or spun filaments, preferably spun filaments, obtained via the method described herein. Additionally provided is a spun mat or spun filaments of crosslinked dialdehyde microfibrillated cellulose. The presence of crosslinks between MFC nanofibrils can be ascertained by spectroscopic methods, e.g. ¹³C NMR.

The spun fibrous material may have improved compatibility with common polymeric matrices, e.g. polyolefins compared to filaments spun from native MFC or other grades of MFC. Therefore, a polymer composite is provided which comprises the spun fibrous material described herein. A method for providing a polymer composite is also provided, said method comprising preparing a fibrous material of crosslinked microfibrillated cellulose according to the invention, and; blending said fibrous material with a polymer matrix to form a polymer composite. The skilled person is aware of standard methods for constructing polymer matrices, and incorporating fibrous material into such matrices.

### EXAMPLES

1. Dry-spinning of DA-MFC+native MFC mixture

Effect of pH

Materials:

DA-MFC+native MFC (DA-MFC/MFC=60%/40%); Degree of Oxidation (DA-MFC)=40%; pH=4.4; ˜1 wt %

Experimental:

The pH of DA-MFC/MFC dispersion was adjusted with either 0.1 M HCl or 0.1 M NaOH to obtain the following pHs: 2.5, 7.2 and 10.6.

Dispersions were then concentrated by centrifugation (Sigma 2-16 KL centrifuge; 10 min at 4350 rpm; no break). The final solids content was about 2 wt % for all samples, except the sample at pH 10.6, which was harder to concentrate using this approach (final solids content 1.2 wt %).

Concentrated DA-MFC/MFC dispersions at different pH were spun directly onto plastic Petri dishes using a 20 mL plastic syringe without needle. Single filaments were created.

The spun filaments were left to dry at ambient conditions (approximately 25° C.).

Upon drying, the strength of the filaments was manually assessed, both in dry and wet state. In the latter case, filaments were immersed in water for about 40 s before being tested.

Observations:

DA-MFC/MFC at pH 10.6 formed thicker filaments when extruded, probably due to the lower solids content. Upon drying, the filaments attached strongly to the Petri dish and it was not possible to detach them. Water was added to the Petri dish in an attempt to detach the filaments, but they became soft and eventually disintegrated.

All the filaments became flat upon drying, probably due to the relatively low solids content.

No significant swelling was observed when the filaments were immersed in water, probably due to the contribution of the less hydrophilic nature of DA-MFC when compared with native MFC.

Mechanical strength (both in dry and wet state), as assessed manually, increased in the order:

(DA-MFC/MFC pH=10.6)<DA-MFC/MFC pH=7.2<DA-MFC/MFC pH=4.4<DA-MFC/MFC pH=2.5

Consequently, filaments from DA- MFC pH 2.5 were strongest which indicates that they have a higher extent of cross-linking.

1. A method for preparing a fibrous material of crosslinked microfibrillated cellulose, said method comprising the steps of:
i. spinning a cellulose composition comprising dialdehyde microfibrillated cellulose (DA-MFC) into a fibrous material; and,
ii. reducing the pH of said fibrous material to pH 7 or below, to provide cross-linking of the dialdehyde microfibrillated cellulose.

2. The method according to claim 1, additionally comprising the step of heat-treatment of said fibrous material, concurrently or subsequently with the step of pH reduction.

3. The method according to claim 2, wherein said heat treatment takes place at a temperature of between 60 and 200° C.

4. The method according to claim 2, wherein said heat treatment takes place for a time of between 10 and 180 minutes.

5. The method according to claim 1, wherein the pH is reduced to below pH 6.5.

6. The method according to claim 1, wherein said fibrous material is filaments.

7. The method according to claim 1, wherein said fibrous material is a mat.

8. The method according to claim 1, wherein said cellulose composition additionally comprises unmodified microfibrillated cellulose.

9. The method according to claim 1, wherein said cellulose composition additionally comprises chemically-modified microfibrillated cellulose.

10. The method according to claim 1, wherein said cellulose composition comprises more than 25% by weight DA-MFC.

11. The method according to claim 1, wherein the crosslinking takes place in the absence of any additional crosslinking agents.

12. The method according to claim 1, wherein the spinning is selected from the group consisting of: wet-spinning, electrospinning and dry-spinning.

13. The method according to claim 1, wherein said dialdehyde microfibrillated cellulose (DA-MFC) is obtained by reacting cellulose pulp fibres with periodate, so as to introduce aldehyde moieties to the cellulose pulp fibres, and subsequently fibrillating said modified cellulose pulp fibres.

14. The method according to claim 1, further comprising the step of drying said fibrous material, before or after step ii.

15. A spun mat or spun filaments obtained via the method of claim 1.

16. A spun mat or spun filaments comprising crosslinked dialdehyde microfibrillated cellulose.

17. A method for preparing a web of spun filaments, said method comprising preparing spun filaments of crosslinked dialdehyde microfibrillated cellulose according to claim 1, and; laying said spun filaments to provide a web.

18. A web comprising spun filaments, wherein said spun filaments are according to claim 15.

19. The web according to claim 18, wherein said web comprises additional filaments or fibres.

20. The web according to claim 18, wherein said web is woven or non-woven.

21. A polymer composite comprising the spun fibrous material according to claim 15.

22. A method for providing a polymer composite, said method comprising:
preparing a fibrous material of crosslinked microfibrillated cellulose according to claim 1; and;
blending said fibrous material with a polymer matrix to form a polymer composite.
